(19) United States
(12) Patent Application Publication
(10) Pub. No.: US 2007/0060653 A1
(43) Pub. Date: Mar. 15, 2007

(54) Physiologic vaginal lubrication to optimize sperm survival and function

(76) Inventor: Ronald J. Thompson, Ft. Thomas, KY (US)

Correspondence Address: Donald N. Halgren, 35 Central Street, Manchester, MA, 01944, US

(21) Appl. No.: 11/407,383

(22) Filed: Apr. 19, 2006

Related U.S. Application Data
(63) Continuation-in-part of application No. 11/123,595, filed on May 7, 2005.
(63) Continuation-in-part of application No. 11/174,037, filed on Jul. 1, 2005.
Application No. 11/174,037 is a continuation-in-part of application No. 09/736,973, filed on Dec. 14, 2000, now abandoned.

(51) Int. Cl. A61K 31/198; A61K 31/045

(52) U.S. Cl. 514/565; 514/729

(57) ABSTRACT

A topical application of a preparation of menthol, or a related cooling compound, and L-arginine to the female clitoris, a reflex normal physiological vaginal lubrication can be induced. This physiological vaginal lubrication provides optimum support for spermatozoa survival and function within the female reproductive tract, specifically in the vagina and cervical mucus. This optimum spermatozoal support is afforded by the biophysical characteristics of the physiological vaginal lubrication, including pH, hydration, oxygenation, and human serum albumin necessary to initiate spermatozoal capacitation. The optimum spermatozoa survival and function in the female reproductive tract should translate to an increase per cycle oocyte fertilization/conception rate in human females.

### BACKGROUND OF THE INVENTION

1. Field of the Invention

This invention relates to methods and pharmaceutical formulations for treating female sexual dysfunction, and more particularly to vaginal, vulvar and/or urethral administration of a vasoactive agent. Cross-reference is made to prior applications and is a continuation in part of U.S. patent application Ser. No. 11/174,037 filed Jul. 1, 2005, and is a continuation-in-part application of my earlier pending U.S. patent application Ser. No. 09/736,973, filed Dec. 14, 2000, entitled “Clitoral Sensitizing Arrangement,” and a continuation-in-part application of U.S. Pat. No. 6,322,493, entitled “Expanded Clitoral Sensitizing Compounds with Methods and Apparatus for the Delivery of these Compounds,” all of which are incorporated herein by reference in their entirety.

2. Background and Prior Art

In 2003, reports from the National (U.S.) Center for Health Statistics and the Centers for Disease Control and Prevention estimated that 75% of infertile women experience vaginal dryness and lubrication insufficiency! The definition of infertility is “one year of regular intercourse, two to three times weekly, with regular periods, without achieving pregnancy.” The recognized authoritative text on infertility is Clinical Gynecologic Endocrinology and Infertility, 6th ed., Baltimore, Md.: Leppincott, Williams, and Wilkins, written by Drs. Speroff, Kase, and Glass. On pages 1013 through 1037, Drs. Speroff, Kase, and Glass report that “As more Americans defer marriage and postpone pregnancy, the number of infertile couples continues to grow. One third of women postponing pregnancy until their mid to late thirties will have an infertility problem, while half of those over age 40 will require medical assistance to conceive.” To graphically display this relationship of a woman's age and fertility/infertility, the graph Female Fertility Over Time is presented from “Seminars in Reproductive Endocrinology,” 1991; 9: 165-175 by Dr. G. B. Maroulis.

[image]

Vaginal lubrication is the initial physiological response of a woman's sexual arousal. The normal vaginal lubrication is actually a plasma transudate from the vascular plexus surrounding a woman's vagina, that diffuses across the vaginal mucosa, and into the lumen of the vagina. Dr. L. Anderson, et al in “The Effects of Coital Lubricants on Sperm Motility in Vitro” stated that “Patients undergoing infertility investigations frequently experience sexual dysfunction, which often is due to inadequate vaginal lubrication.” Lubrication insufficiency, the inability to adequately vaginally lubricate, could be related to the sexual dysfunction of infertility, or could be related to a woman's age, since the vast majority of infertile women are over age 30.

Commercially available replacement vaginal lubricants, such as KY Jelly, Replens, Astroglide, and Femglide have all proven to decrease sperm motility in vitro and some are even spermicidal! Because of numerous medical articles reporting the adverse actions of vaginal lubricants on sperm, it is currently contraindicated for infertility patients to use any vaginal lubricants to treat insufficiency. In “Vaginal Lubricants for the Infertile Couple: Effects of Sperm Activity,” by Kutteh et al from the International Journal of Fertility and Menopausal Studies, 1996 July-August; 41(4): 400-4, Dr. Kutteh reports “Commercial lubricants inhibited sperm motility by 60-100% after 60 minutes of incubation. Sperm exposed to Replens or Astroglide were nonmotile and nonviable after incubation for 60 minutes.”

U.S. Pat. No. 6,140,121 filed Oct. 17, 1996 and issued Oct. 31, 2000, “Methods and Composition to Improve Germ Cell and Embryo Survival and Function,” describes an intravaginal coital lubricant that does not decrease sperm motility or sperm viability. Subsequent U.S. Pat. No. 6,593,309, filed Feb. 28, 2000 and issued Jul. 15, 2003, and published U.S. patent application 20040073964, filed May 7, 2003 and published Apr. 15, 2004 further describe and define the only coital lubricant that is actually recommended for infertility patients. The important fact is that the described product Pre-seed, does not decrease sperm motility or viability.

U.S. publishes patent application number 20050244520 “Topical menthol, or a related cooling compound, to induce lubrication,” filed Jul. 1, 2005 and published Nov. 3, 2005 teaches of the reflex vaginal lubrication in the vagina, as a reflex reaction to the stimulation of the clitoris by menthol or a related cooling compound. This published application anticipates the use of topical clitoral menthol, or menthol and L-arginine, to treat lubrication insufficiency, but not relative to infertility, only relative to sexual dysfunction.

### Sperm Survival and Function

Dr. R. J. Levin in his article entitled “The physiology of sexual arousal in the human female: a recreational and procreational synthesis,” Archives of Sexual Behavior, 2002 October; 31(5): 405-11, insightfully states “Sexual arousal initiates enhanced genital blood flow, leading to the formation of a neurogenic transudate, lubricating the vagina, partly buffering its acidity, and increasing its oxygen tension all features that enhance spermatozoal function and survival.” This describes the normal physiological vaginal lubrication produced by sexual arousal, actually has a purpose to support, nurture, and optimize sperm survival and function to optimize fertility. These sperm support functions include decreasing hostile vaginal acidity, increasing vaginal oxygenization, increasing cervical hydration, and providing a source of albumin for the cervical mucus to initiate capacitation of sperm. The normal physiologic vaginal lubrication is a transudate of blood. This transudate is actually the fluid component of blood, the plasma, devoid of all cellular components such as platelets, white blood cells, and red blood cells. This transudate reflects the physical characteristics of blood, pH 7.4, 70-80% water, the source of hydration, 99% oxygen saturation, and the source of albumin. It is most important to understand that all of these important physical properties of the vaginal luminal transudate will be absorbed by the cervical mucus by simple diffusion. The more abundant the vaginal transudate, the more neutralized the normal vaginal pH of 2.5 to 4.5. (Sperm cannot survive below a pH of 6.0) The more abundant the vaginal transudate the higher the oxygen level of the vaginal lumen. The more abundant the vaginal transudate, the higher the water and albumin concentrations of the cervical mucus will be.

### Cervical Mucus Acidity

Drs. Zavos and Cohen reported in “The pH of cervical mucus and the post-coital test,” published in Fertility and Sterility, 1980 September: 34(4), 234-8 that “The results obtained revealed significant differences (P<0.01) between the low pH (less than or equal to 6) and the high pH (greater than or equal to 6.1) groupings with respect to percentage motility and grade of recovered spermatozoa (from the post coital cervical mucus). Drs. Peek and Matthews confirmed this and concluded “All spermatozoa were immobile in the post-coital test when the pH of the cervical mucus was below 6.” This was reported in an article entitled “The pH of cervical mucus, quality of semen, and outcome of the post-coital test,” published in Clinical Reproductive Fertility, 1986 January; 4(3): 217-25. Both of these references confirm the pH of the cervical mucus as an important factor in the optimum sperm survival. The pH of the cervical mucus depends upon multiple factors but certainly the most important is the pH of the vagina. The normal vaginal pH is 2.5 to 4.5. The pH of the physiologic vaginal transudate is that of blood, 7.4. The more abundant the physiological vaginal transudate, the higher the vaginal pH will become during sexual arousal. The higher the vaginal pH, and the more abundant luminal transudate, the more likely that the cervical pH will be above 6.0, due to passive diffusion from the luminal transudate to the cervical mucus.

### Cervical Mucus Hydration (And Oxygen Dissolved in the Water)

“Under the influence of estrogen the cervix secretes highly hydrated mucus, often exceeding 96% water in women,” reported Dr. Katz, et al in “analysis of preovulatory changes in cervical mucus hydration and sperm penetrability,” published in Advanced Contraception, 1997, 13, 143-51. Dr. Morales et al reported that “The extent of hydration is correlated with permeability to sperm,” in Human Reproduction (1993) 8, 78-83 in an article entitled “Human cervical mucus: Relationship between biochemical characteristics and ability to allow migration of spermatozoa.” Both of these references related the importance of cervical mucus hydration and the ability of spermatozoa to penetrate the cervical mucus. The cervical mucus is produced in the endocervical glands. Hydration of the cervical mucus adjacent to these endocervical glands is a function of the estrogen status of the woman, but hydration of the cervical mucus in the central lumen of the endocervix is dependent upon the absorption of water from the vaginal lumen. The more abundant the vaginal transudate, the more water to hydrate the cervical mucus in the lumen of the endocervix.

The importance of cervical mucus hydration, and therefore sperm penetration of the cervical mucus into the uterus and fallopian tube to encounter an oocyte to accomplish fertilization and establish a pregnancy is stated by Dr. Bigelow. “Coitus on the day of maximal mucus hydration in women is more closely correlated with incidence of pregnancy than coitus timed with respect to ovulation using basal body temperature.” This conclusion was presented by Dr. Bigelow et al in “Mucus observations in the fertile window: a better predictor of conception that the timing of intercourse,” published in Human Reproduction, (2004) 19, 889-92.

### Cervical Mucus Proteins and Glycoproteins

As previously described, the normal physiological vaginal lubrication associated with a woman's sexual arousal is a vaginal plasma transudate. Plasma is the liquid component of blood that contains water, buffering ions, dissolved oxygen and proteins, and glycoproteins. Glycoproteins are entities that combine both proteins and sugars. Plasma by definition contains proteins that are coagulation factors. Serum is the blood plasma, with coagulation factors removed. The normal plasma transudate contained in the vaginal lumen contains all of the components of human serum albumin (HAS) except the coagulation factors. This is important because the human serum albumin is essential for sperm capacitation, a transformation of the sperm that allows the sperm to both access and fertilize the oocyte. The human serum albumin in the vaginal transudate will, like the ions for pH determination, the water for hydration, and the dissolved oxygen for oxygenation, passively diffuse from the vaginal lumen into the cervical mucus. This diffusion from the vaginal lumen to the cervical lumen is dependent upon the volume of vaginal plasma tranduate and the volume of cervical mucus. Passive diffusion is a process of movement from an area of high concentration to an area of low concentration. Therefore, the more abundant the plasma transudate in the vaginal lumen, the more efficient will be the movement of the ions, water, oxygen, and human serum albumin into the cervical mucus. Human serum albumin is the most important of these factors because only a capacitated sperm can fertilize an oocyte and establish pregnancy.

### Sperm Capacitation

Dr. L. R. Fraser in “Sperm capacitation and the acrosome reaction,” Human Reproduction 1998 April; 13 Supplement 1: 9-19, defines sperm capacitation with the statement, “To achieve successful fertilization under normal circumstances in vivo, mammalian spermatozoa must first undergo capacitation and then the acrosome reaction, an exocytotic event that allows cells to penetrate the zona pellucida and fuse with the oocyte plasma membrane.” In the Indian Journal of Experimental Biology, 2005 November; 43(11): 1001-15, Drs. Mitra and Shivaji published a review entitled “Proteins implicated in sperm capacitation.” Drs. Mitra and Shivaji state, “Spermatozoa after being deposited in the female reproductive tract spend a considerable time in this foreign environment prior to fertilization of the oocyte.” Chang and Austin independently observed (in 1951) that this time spent by the spermatozoa in the female tract is not consequential but a necessary event in the life cycle of the male gamete.” Sperm capacitation is only accomplished by incubation of the sperm with human serum albumin in vitro in preparation of the sperm for artificial insemination or for in vitro fertilization. Sperm capacitation in vitro with human serum albumin has been the gold standard of care for the past twenty years.

In vivo sperm capaciation is not yet completely understood. What is understood, is the action of the proteins of the human serum albumin to chelate, or strip the cholesterol (sterol) molecules off the surface of the ejaculated spermatozoa. This cholesterol is a normal component of seminal plasma, in which the individual spermatozoa are suspended in the ejaculate. Dr. C. Dejonge, in his 2005 review article “Biological basis for human capacitation,” published in Human Reproduction Update, Vol. 11, No. 3, pp. 205-214 states “This cervical mucus is demonstrated to have a capacitating influence that is augmented in the presence of serum albumin.” Dr. DeJonge further states that in vitro capacitation, “the media must contain a sterol-acceptor molecule, e.g. serum albumin.”

The essential presence of human serum albumin to initiate the sperm capacitation is widely reported in the literature. With an increase of vaginal transudate lubrication, one would expect an increase in the serum albumin in the cervical mucus by passive diffusion from an area of high concentration, the vaginal lumen, to an area of low serum albumin concentration, the cervical mucus. Dr. M. S. Scott in a review article, “A glimpse at sperm function in vivo: sperm transport and epithelial interaction in the female reproductive tract,” published in the 2000 Animal Reproductive Science, July 2; 60-61: 337-48 concludes, “The dynamic interactions that occur between functional spermatozoa and the luminal fluids and the epithelial surfaces of the female genital tract during transit and storage enhance sperm survival and regulate sperm function in the female.” (Luminal fluids are vaginal transudate, endocervical mucus, endometrial secretions, in the uterus, and endosalpinx secretions).

Sperm capacitation is the transformation of the sperm described as flagellar (tail) whip-like motions that allows for forward propagation of the spermatozoa and the acrosome reaction. Both are initiated by the serum albumin dilation of the spermatozoa surface cholesterols. The acrosome reaction is the organization of the head of the sperm into a cap that contains enzymes to digest the cumulus oopherus, zona pellicude, and oocyte cell membrane. This digestion allows entrance of the nuclear DNA of the spermatozoa into the nucleus of the oocyte, or fertilization. In “acrosome intactness and seminal hyaluronidase activity: relationship with conventional seminal parameters: Dr. Tanble et al state that “seminal hyaluronidase activity, thus determined, is primarily dependent upon the intact status of the acrosome.” This is published in the Indian Journal of Medical Science. 2001 March; 55 (3): 125-32. The initiation of spermatozoa capacitation and acrosome reaction are quite complex, but completely dependent upon human serum albumin. In this abstract by Dr. Lefievre et al, the source of the serum albumin is fetal cord serum ultrafiltrate:
Lefievre et al., “Activation of Protein Kinase A During Human Sperm Capacitation and Acrosome Reaction,” J. Androl. 2002 September-October; 23 (5): 709-16.
Spermatozoa undergo a variety of changes during their life that are prerequisites to their maturation and ability to fertilize eggs. Mammalian sperm capacitation and acrosome reaction are regulated by signal transduction systems involving cyclic adenosine monophosphate (cAMP) as a second messenger. This second messenger acts through the activation of protein kinase A (PKA) and directly regulates protein tyrosine phosphorylation. cAMP levels are controlled by a balance of phosphodiesterases (PDEs) and adenylyl cyclase (AC) enzymatic activities, which are responsible for its degradation and production, respectively. The aim of this study was to evaluate the possible relationship between the intracellular levels of cAMP and PDE and PKA activities during human sperm capacitation induced by fetal cord serum ultrafiltrate (FCSu) and acrosome reaction induced by calcium ionophore A23187. We report that spermatozoa and the intracellular levels of cAMP decreased but that PDE induced by A23187 was associated with increases in cAMP and PKA activity but not in PDE activity. These results strongly suggest that net cAMP concentration is under the control of AC, since PDE activity is constant during sperm capacitation and the acrosome reaction. Moreover, the results suggest that low levels of cAMP are sufficient for capacitation and PKA activation and/or that the cAMP concentration measured in whole spermatozoa does not reflect the effective intracellular cAMP levels present in specific compartments of these cells.

### Spermatozoa Storage and Transport in the Female Reproductive Tract

Tens to hundreds of million individual spermatozoa are deposited in the anterior vagina during coitus. A vast number of these spermatozoa are lost though the vaginal introitis, but a pool of spermatozoa suspended in seminal fluid and vaginal plasma transudate, remain in the posterior vaginal fornix. The external cervical os and extruding cervical mucus are immersed in the pool of spermatozoa, seminal fluid and vaginal plasma transudate. Spermatozoa both diffuse into and actively swim into the cervical mucus for the length of time that the spermatozoa remain viable in the vagina. Dr. Hafez in “In vivo and in vitro sperm penetration in cervical mucus.” Acta European Fertility 1979 January; 10 (2): 41-9, reports that “Spermatozoa are immobilized by vaginal secretions within one to two hours of insemination.” Dr. Hafez also describes “Sperm transport in the female reproductive tract involves 3 stages: rapid short sperm transport; colonization of reservoirs; and the slow prolonged release (of spermatozoa).” It is the reservoir function of the endocervical crypts and the endocervical mucus that allows for a constant supply of capacitated spermatozoa for a 24 to 36 hour window after coitus. This supply of spermatozoa is to the distal fallopian tubular ampula where oocyte penetration and fertilization occur.

The post-coital test (Sims-Huhner Test) is a test of spermatozoa concentration in the cervical mucus 6 to 12 hours after coitus. Fifteen to twenty motile active spermatozoa per high power field (HPF) after evaluating, at least 10 HPF's, is a very satisfactory post-coital test of spermatozoa storage and support in the cervical mucus. The reservoir function of the cervical mucus and the spermatozoa capacitating function of the cervical mucus ultimately dictate success in fertilizing the oocyte and ultimately in establishing a pregnancy. Dr. Hall et al published in the British Journal of Obstretics and Gynecology, 1982 April; 89 (4): 299-305, “Prognostic value of the post-coital test: Prospective study based on time-specific conception rates.” Dr. Hall concluded, “The time to conception was inversely related to the number of motile sperm seen. Simpler analysis slowed a five fold greater chance of conception associated with a positive compared to a negative post-coital test; after 2 years the cumulative conception rates were 82% and 16% respectively.” This substantiates the importance of the cervical mucus to achieve pregnancy.

### DESCRIPTION OF THE PRESENT INVENTION

U.S. published patent application 20050244520, “Topical Menthol, or any related cooling compound, to Induce Lubrication,” Filed Jul. 1, 2005 and published Nov. 3, 2005, and incorporated herein by reference, describe the reflex production of normal physiological vaginal lubrication as a treatment for the disorder decreased sexual arousal compound of female sexual dysfunction. Although the title only describes menthol and related cooling compounds, the text also defines a combination of menthol, or a related cooling compound, and L-arginine topically applied to the clitoris to evoke a reflex vaginal lubrication. U.S. Pat. Nos. 6,332,493 and 6,702,733, incorporated herein by reference, both teach of the topical use of the above described combination product of menthol, or a related cooling compound, and L-arginine, but not as an adjunct therapy for fertility.

This patent defines the use of the topical menthol, or a related cooling compound, in combination with L-arginine, to be used to treat lubrication insufficiency ascribed to 75% of infertile women. Furthermore, this patent describes and defines how the induction of a physiological vaginal transudate by the topical clitoral application of the menthol, or a related cooling compound, and L-arginine, can support the function and viability of the spermatozoa, initially in the hostile vaginal environment, and ultimately in the cervical mucus. The bio-physical properties of the normal physiological vaginal transudate transferred to the cervical mucus allow for the bio-physical optimum survival and function of the spermatozoa in the cervical mucus. These bio-physical properties are ions for a favorable pH above 6.0, water for cervical mucus hydration, and the oxygen dissolved in this hydration for oxidative support of the spermatozoa, and the human serum albumin necessary to capacitate the spermatozoa. By maximizing all of these spermatozoa supporting bio-physical properties transferred from the physiological vaginal transudate to the cervical mucus, this will optimize spermatozoa survival and function in the female reproductive tract. By optimizing spermatozoa survival and function in the female reproductive tract, the odds of conception, per cycle, or cumulatively over a defined period of time as Dr. Hall stated, should increase, to increase the conception rate.

### BRIEF DESCRIPTION OF THE FIGURES

Referring now to FIGS. 1-9, conveniently labeled for descriptive purposes and for reference, are as follows:

FIG. 1 represents a Spermatozoa, showing its head with its DNA therein, and its tail portion showing a coating of cholesterol thereon;

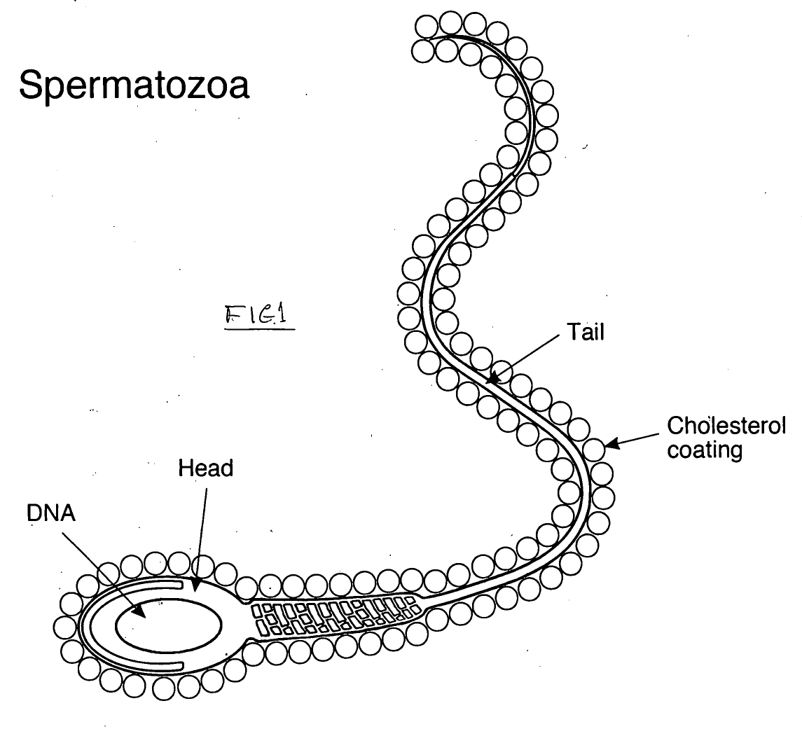

FIG. 2 represents Capacitation I stage, with the Spermatozoa entering the Vaginal transudate lubrication, with the Human serum albumin initiating the dissolution of the cholesterol coating off of the spermatozoa;

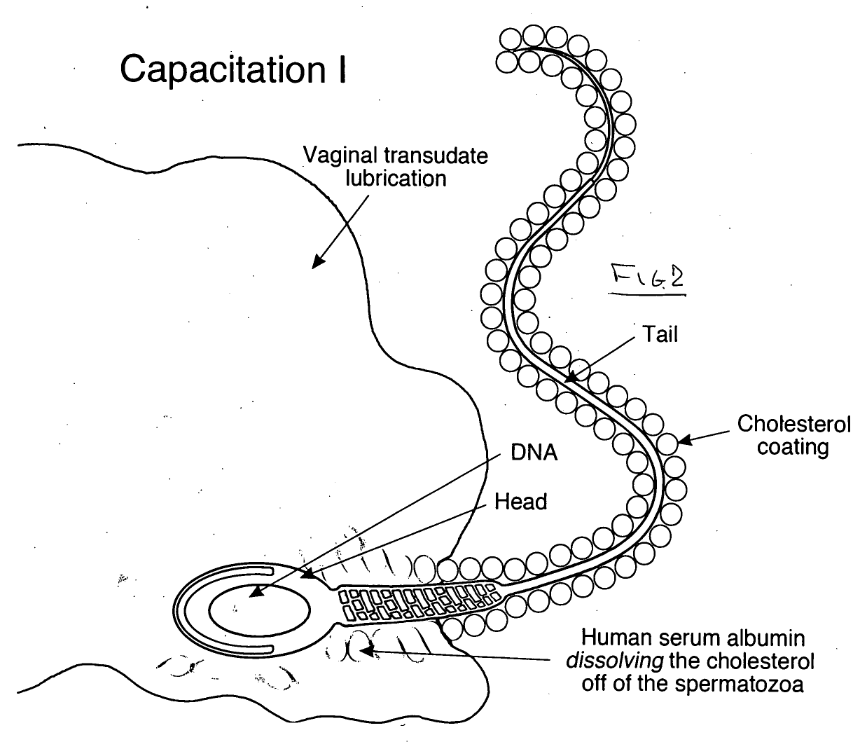

FIG. 3 represents the Capacitation II stage, with the Vaginal transudate lubrication, that is, the human serum albumin in the cervical mucus completely dissolving away all of the cholesterol from the spermatozoa, thus permitting the tail thereof to initiate its whip-like action for forward motion of that spermatozoa;

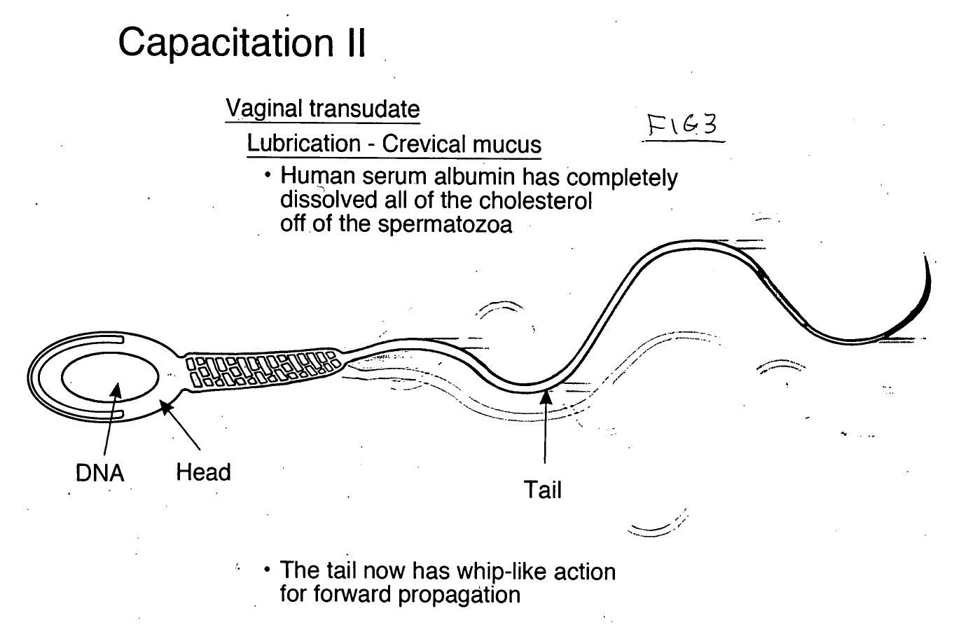

FIG. 4 represents the Capacitation III stage, wherein the Acrosome, which is a cap-like structure on the head of the spermatozoa, which structure contains enzymes to digest and dissolve the protective coverings of an oocyte, the tail of the spermatozoa trailing in the vaginal transudate—the cervical mucus;

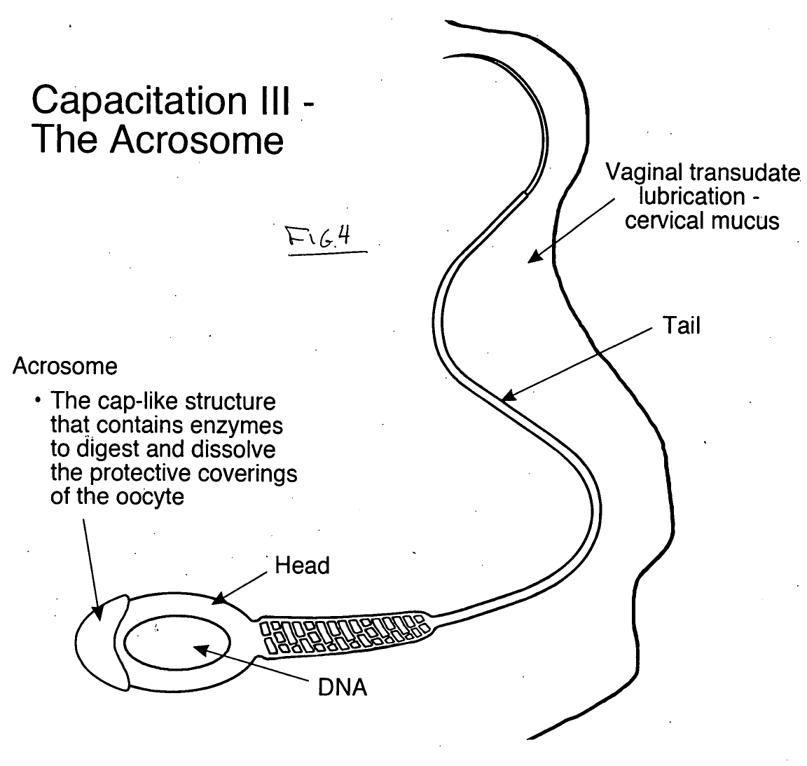

FIG. 5 represents the Oocyte I stage with a head (containing male DNA) of a single Spermatozoa pressing against and digesting and dissolving the oocyte cell membrane by the Acrosome enzymes thereon;

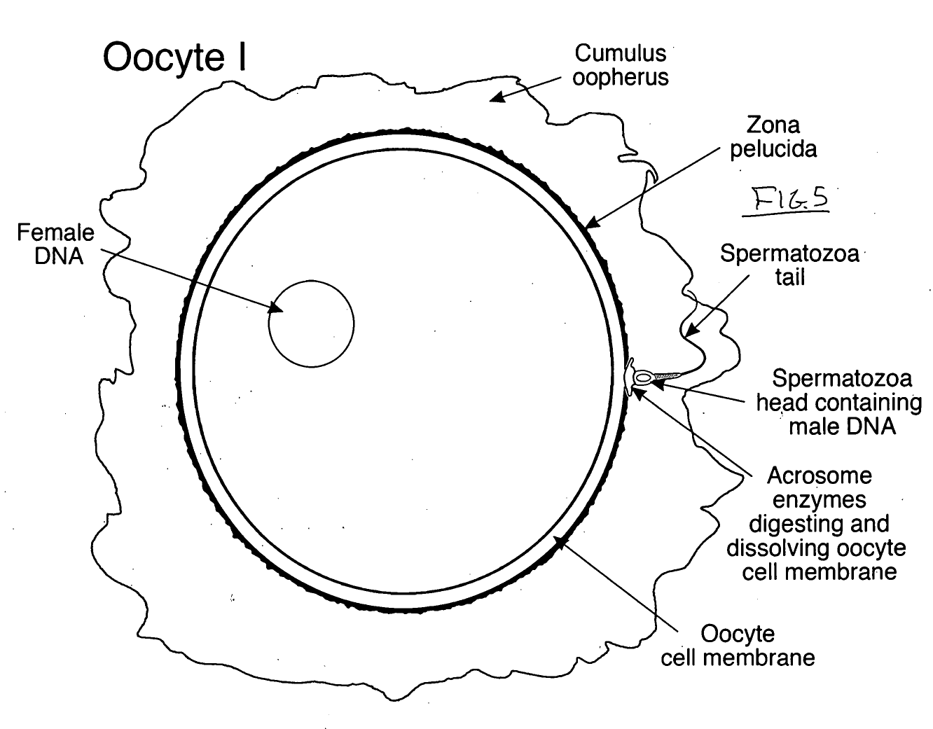

FIG. 6 represents the Oocyte II stage wherein the spermatozoa has entered through the dissolved opening in the cell membrane and propelled its way into the oocyte, the tail still whipping within the Cumulus oopherus surrounding the Oocyte;

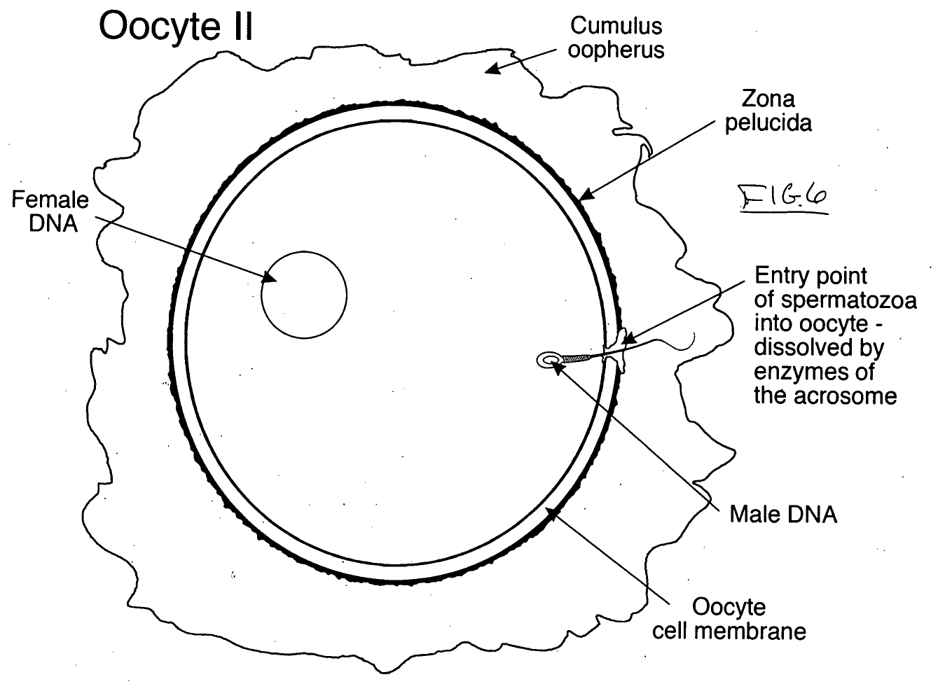

FIG. 7 represents the Cervix, in a lateral view, with the spermatozoa immersed in vaginal transudate lubrication, pooled in the cul-de sac of the vagina, with the transudate supplying the cervical mucus with ions to decrease acidity, water to hydrate the cervical mucus, oxygen and human serum albumen for the capacitation process;

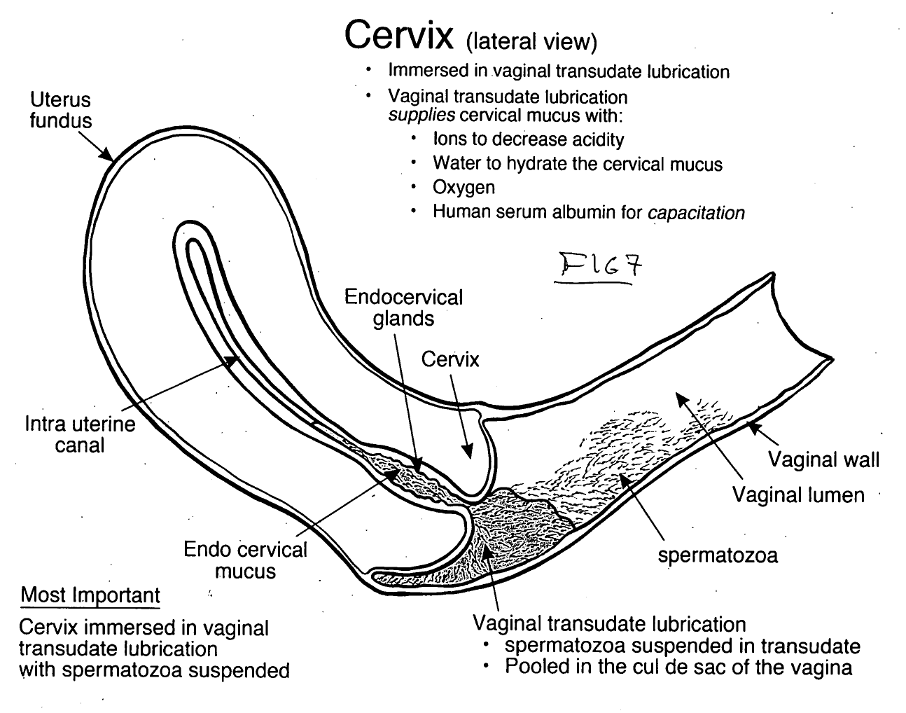

FIG. 8 represents the Cervix, in a frontal view, with the spermatozoa shown immersed in the vaginal transudate lubrication; and

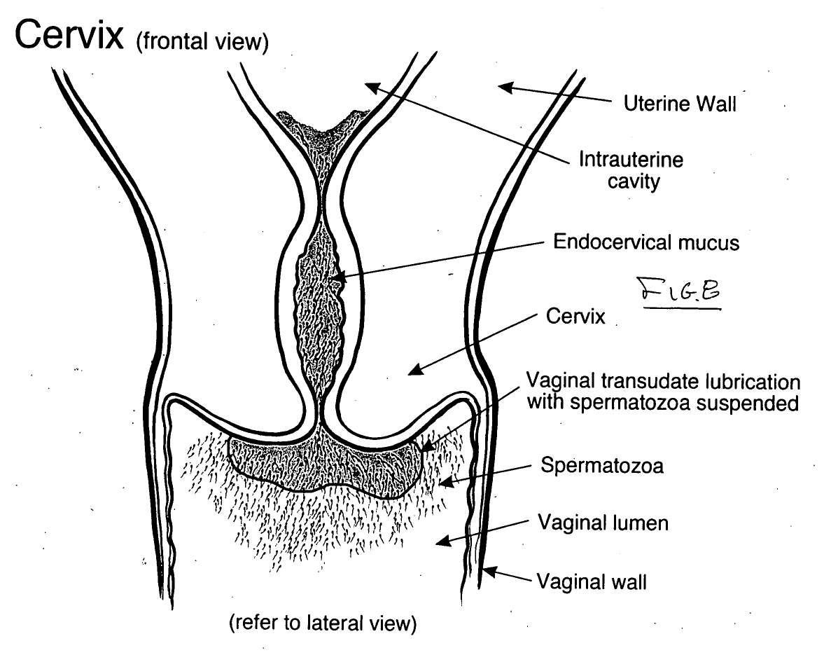

FIG. 9 representing the fertilization occurring in the ampulla of the fallopian tube, from the ovulation or release of the oocyte, through day one which is the fertilization of oocyte in ampulla of fallopian tube (oviduct), day two through day six representing the cell replication within the intrauterine cavity.

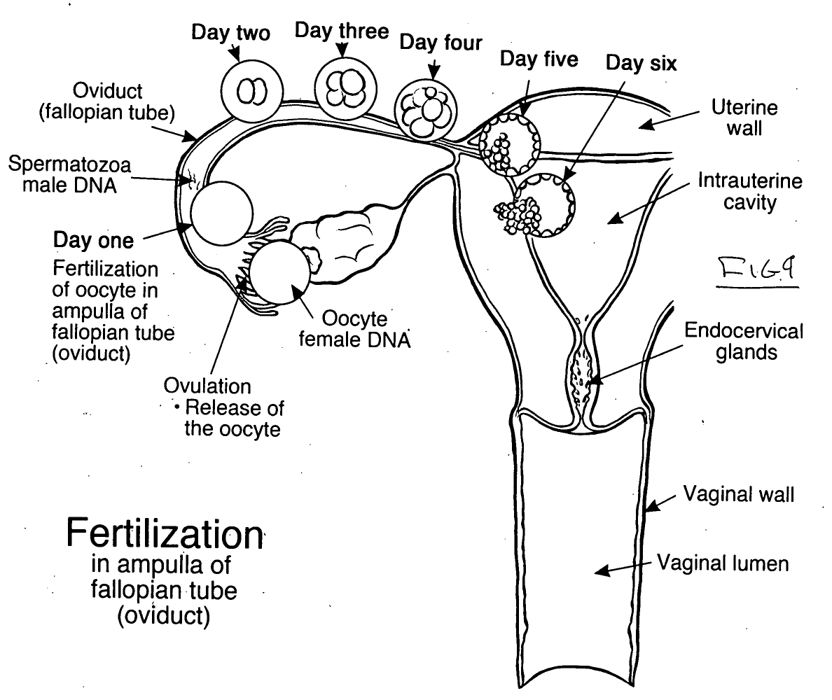

### BRIEF SUMMARY OF THE INVENTION

The invention thus comprises a method of inducing physiological vaginal lubrication in a mammalian female by applying a compound from a reservoir, directly onto the vagina, vulva or urethra of that female said compound comprising an admixture of menthol and L-arginine. The menthol and said L-arginine may each comprise less then 5% of said compound. The invention includes a method of rendering the vaginal luminal environment of a mammalian female less acidic, by applying a compound from a reservoir onto the vagina, vulva or urethra of that female said compound comprising an admixture of menthol and L-arginine. The invention also includes a method to supply the substrate of proteins and glycoproteins of human serum albumin to the vaginal lumen of a female to initiate capacitation and increase the spermatozoal survival and function in vivo in the vagina, by the step of: applying a compound from a compound reservoir, directly onto the vagina, vulva or urethra of that female, said compound comprising an admixture of menthol and L-arginine. The invention also includes a method to supply the substrate of proteins and glycoproteins of human serum albumin to the cervical mucus of a female, to support capacitation and increase spermatozoal survival and function in vivo in the cervical mucus, by: applying a compound from a compound reservoir, directly onto the vagina, vulva or urethra of that female, said compound comprising an admixture of menthol and L-arginine. The invention thus also includes a method to treat lubrication insufficiency in the vaginal lumen, without using any preparations, comprising: applying a topical clitoral menthol, or a related cooling compound to the vagina, to induce a normal physiological vaginal lubrication therein. The invention includes a method to render the endocervical mucus less acidic and therefore more receptive to spermatozoa by improving cervical mucus survival and function of spermatozoa in vivo, by: applying a topical compound of clitoral menthol, or a related cooling compound to the vagina of a female, and a nitric oxide donor to induce a normal physiological vaginal lubrication. The nitric oxide donor may comprises nitroglycerine.

I claim:

1. A method of inducing physiological vaginal lubrication in a mammalian female by:
applying a compound from a reservoir, directly onto the vagina, vulva or urethra of that female said compound comprising an admixture of menthol and L-arginine.

2. The method as recited in claim 1, wherein said menthol and said L-arginine each comprise less then 5% of said compound.

3. A method of rendering the vaginal luminal environment of a mammalian female less acidic, by:
applying a compound from a reservoir onto the vagina, vulva or urethra of that female said compound comprising an admixture of menthol and L-arginine.

4. A method to supply the substrate of proteins and glycoproteins of human serum albumin to the vaginal lumen of a female to initiate capacitation and increase the spermatozoal survival and function in vivo in the vagina, by the step of:
applying a compound from a compound reservoir, directly onto the vagina, vulva or urethra of that female, said compound comprising an admixture of menthol and L-arginine.

5. A method to supply the substrate of proteins and glycoproteins of human serum albumin to the cervical mucus of a female, to support capacitation and increase spermatozoal survival and function in vivo in the cervical mucus, by:
applying a compound from a compound reservoir, directly onto the vagina, vulva or urethra of that female, said compound comprising an admixture of menthol and L-arginine.

6. A method to treat lubrication insufficiency in the vaginal lumen, without using any preparations, comprising:
applying a topical clitoral menthol, or a related cooling compound to the vagina, to induce a normal physiological vaginal lubrication therein.

7. A method to render the endocervical mucus less acidic and therefore more receptive to spermatozoa by improving cervical mucus survival and function of spermatozoa in vivo, by:
applying a topical compound of clitoral menthol, or a related cooling compound to the vagina of a female, and a nitric oxide donor to induce a normal physiological vaginal lubrication.

8. The method as recited in claim 7, wherein said nitric oxide donor comprises nitroglycerine.
